(19) United States
(12) Patent Application Publication
(10) Pub. No.: US 2009/0259280 A1
(43) Pub. Date: Oct. 15, 2009

(54) ELECTRICAL STIMULATION LEAD WITH BIOERODIBLE ANCHORS AND ANCHOR STRAPS

(76) Inventors: Kevin Wilkin, Valencia, CA (US); Jeryle Walter, Valencia, CA (US); Timothy Raymond Odell, Los Angeles, CA (US); Jeremy Koff, Studio City, CA (US); Morten Hansen, Valencia, CA (US); Arkady Glukhovsky, Santa Clarita, CA (US); Timothy Deer, Charleston, WV (US); Julie S. Armstrong, Glendale, CA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/251,008

(22) Filed: Oct. 14, 2008

Related U.S. Application Data
(60) Provisional application No. 60/980,039, filed on Oct. 15, 2007.

(51) Int. Cl. A61N 1/05

(52) U.S. Cl. 607/116

(57) ABSTRACT

An apparatus includes a coupling portion, a bioerodible anchor portion and a bioerodable retainer. The coupling portion is configured to be coupled to an electrical conductor. The bioerodible retention portion is adjacent to the coupling portion and is moveable from a collapsed configuration to an expanded configuration. The bioerodible retention portion is configured to anchor the electrical conductor with respect to body tissue when the bioerodible retention portion is in its expanded configuration. The bioerodible anchor portion is formulated to erode when disposed within the body tissue at a first rate. The bioerodible retainer is coupled to the bioerodible anchor portion and is configured to inhibit movement of the bioerodible anchor portion from the collapsed configuration to the expanded configuration. The bioerodible retainer is formulated to erode when disposed within the body tissue at a second rate greater than the first rate.

### CROSS-REFERENCE TO RELATED APPLICATIONS

This application claims priority to U.S. Patent Application No. 60/980,039, entitled “Electrical Stimulation Lead with Bioerodible Anchors and Anchor Straps” filed Oct. 15, 2007, which is incorporated herein by reference in its entirety.

### BACKGROUND

The invention relates generally to a medical device, and specifically to an improvement for an electrical conductor for conveying electrical current to a target tissue in a body of a subject.

Known electrical conductors, also referred to herein as “medical leads” or “implantable leads,” are used for various indications. For example, medical leads are used for electrical stimulation or blocking of activation of a target body tissue, delivery of electrical current to an implanted electrical device, and delivery of an electrical signal from a target body tissue to an external electrical device. Thus, known medical leads provide an electrical connection between components, such as between a device external to the body of the subject and a target body tissue or between an implanted stimulator and a target body tissue. For example, a medical lead can provide an electrical connection between an external stimulator and a nerve in the body of the subject, or between an external electromyogram (EMG) sensor and the nerve. For example, a known medical lead can provide an electrical connection between an external device and a device implanted in the body of the subject, such as an external stimulator and an amplifier implanted in the body of the subject.

Over time, the electrical conductor disposed within the body can become encapsulated by the surrounding tissue, creating a protective barrier against migration. Attempts have been made to reduce migration of the electrical conductor prior to encapsulation by using various known anchoring mechanisms. Such known anchoring mechanisms, however, can prevent and/or inhibit the repositioning and/or removal of the electrical conductor from the body. The electrical conductor may need to be repositioned or removed for various reasons, such as, for example, due to improper placement, irritation, infection, or disuse. Moreover, removal of such known anchoring mechanisms may cause a portion of the deployed anchor to be pulled through the target body tissue or other body tissue when the electrode or electrical conductor is removed from the tissue, causing tissue damage.

Additionally, the force required to dislodge the electrode or electrical conductor increases with time after implantation within the body, due to the development of fibrous tissue encapsulation. The force required to rupture the encapsulation tissue can increase the likelihood of tissue damage during removal of the electrical conductor for the body tissue associated with the known anchoring mechanisms. Furthermore, the force required to rupture the encapsulation tissue may cause breakage of anchoring components, leaving unwanted remnants lodged in the tissue.

Often, known electrical conductors are coupled to a fixation mechanism, such as a tissue anchor, via an interference fit. In certain instances, it is desirable to construct the electrical conductor from a silicone rubber and the tissue anchor from polypropylene, which are not easily bonded together easily. Thus, the coupling between some known tissue anchors and some known electrical conductors can become weakened, thereby disadvantageously resulting in migration of the electrical conductor within body tissue. Moreover, in some application, it is desirable for an electrical conductor to be anchored to body tissue without having a tissue anchor contacting a conductive portion of the electrical conductor.

Thus, a need exists for a passive electrical conductor configured to reduce migration of the electrical conductor prior to encapsulation by surrounding bodily tissue, and thus avoiding a need for further surgical intervention to reposition the electrical conductor in the body of the subject. A need also exists for a fixation mechanism capable of anchoring the electrical conductor and minimizing tissue damage upon its repositioning or removal. A further need exists for a fixation mechanism that can be deployed after a desired position within the tissue has been determined.

A need also exists for improved mechanisms and methods for coupling a tissue anchor to an electrical conductor. For example, a need exists for a tissue anchor that can be coupled to an electrical conductor without contacting a conductive portion of the electrical conductor.

### SUMMARY

Apparatus and methods for anchoring an implantable lead within a body are described herein. In some embodiments, an apparatus includes a coupling portion, a bioerodible anchor portion and a bioerodable retainer. The coupling portion is configured to be coupled to an electrical conductor. The bioerodible retention portion is adjacent to the coupling portion and is moveable from a collapsed configuration to an expanded configuration. The bioerodible retention portion is configured to anchor the electrical conductor with respect to body tissue when the bioerodible retention portion is in its expanded configuration. The bioerodible anchor portion is formulated to erode when disposed within the body tissue at a first rate. The bioerodible retainer is coupled to the bioerodible anchor portion and is configured to inhibit movement of the bioerodible anchor portion from the collapsed configuration to the expanded configuration. The bioerodible retainer is formulated to erode when disposed within the body tissue at a second rate greater than the first rate.

### BRIEF DESCRIPTION OF THE DRAWINGS

FIG. 1A-1D are schematic illustrations of a tissue anchor according to an embodiment of the invention.

FIG. 2 and 3 are perspective views of a tissue anchor, according to an embodiment of the invention in a first position and a second position, respectively.

FIG. 4 is a perspective view of a medical device including an electrical conductor coupled to a tissue anchor, according to an embodiment of the invention.

FIG. 4A is a cross-sectional view of the medical device of FIG. 4 taken along line 4A-4A.

FIG. 5 is a side view of the tissue anchor of FIG. 4.

FIG. 6 is a cross-sectional view of the tissue anchor of FIG. 5 taken along line 6-6.

FIG. 7 is a cross-sectional view of a tissue anchor, according to an embodiment of the invention.

FIG. 8 is a side view of a medical device including an electrical conductor coupled to a tissue anchor, according to an embodiment of the invention.

FIG. 9 and 10 are a front view and a top view, respectively, showing internal components of the tissue anchor of FIG. 8.

FIG. 11 is a side view of a medical device including an electrical conductor and a tissue anchor, according to an embodiment of the invention.

FIG. 12 is a front view of the tissue anchor of FIG. 11.

FIG. 13 is a side view of a medical device according to an embodiment of the invention.

FIG. 14A-14J are schematic illustrations of a medical device being inserted into a body of a subject, according to an embodiment of the invention.

### DETAILED DESCRIPTION

Apparatus and methods for anchoring an implantable lead within a body are described herein. In some embodiments, a tissue anchor includes a coupling portion, a bioerodible anchor portion and a bioerodable retainer. The coupling portion is configured to be coupled to an electrical conductor, such as, for example, an implantable lead. The bioerodible retention portion is adjacent to the coupling portion and is moveable from a collapsed configuration to an expanded configuration. The bioerodible retention portion is configured to anchor the electrical conductor with respect to body tissue when the bioerodible retention portion is in its expanded configuration. The bioerodible anchor portion is formulated to erode when disposed within the body tissue at a first rate. The bioerodible retainer is coupled to the bioerodible anchor portion and is configured to inhibit movement of the bioerodible anchor portion from the collapsed configuration to the expanded configuration. The bioerodible retainer is formulated to erode when disposed within the body tissue at a second rate greater than the first rate.

In some embodiments, the tissue anchor can be coupled to an electrical conductor via an adhesive disposed between the tissue anchor and an outer surface of the electrical conductor. For example, the tissue anchor can define a lumen configured to receive the electrical conductor. The tissue anchor can also define a recess that can receive the adhesive therein such that adhesive contacts an inner wall of the recess and a surface of the electrical conductor when the electrical conductor is received within the lumen. Alternatively, in other embodiments the tissue anchor can be coupled to an eyelet of the electrical conductor or via a suture coupled to the electrical conductor via an adhesive, weld, interference fit, or the like.

In another embodiment, a tissue anchor includes a retention portion and a coupling portion. The retention portion is configured to anchor an electrical conductor with respect to body tissue. The coupling portion is configured to be coupled to the electrical conductor. The coupling portion defines a lumen therethrough. The lumen is configured to receive at least a portion of the electrical conductor. The coupling portion further defines a recess configured to receive an adhesive therein. The adhesive is configured to maintain the coupling portion in a position relative to the electrical conductor.

In yet another embodiment, an apparatus includes an electrical conductor and a tissue anchor. The electrical conductor includes a conductor portion and an eyelet portion. The tissue anchor is coupled to the electrical conductor and is spaced apart from an outer surface of the electrical conductor. For example, in some embodiments, the electrical conductor can have an insulating member (e.g., a silicone sheath) and the anchor can be coupled to the insulating member rather than directly to the electrical conductor. The tissue anchor includes a body portion having a first diameter and a retention portion having a second diameter greater than the first diameter. The retention portion is configured to anchor the electrical conductor with respect to body tissue.

As used herein, the term bioerodible means capable of being degraded, dissolved, absorbed and/or disassembled, or digested by action of a biological environment. The action of a biological environment includes the action of living organisms, exposure to a substance having a physiological pH, a change in temperature, and electrical stimulation. For example, a portion of a tissue anchor can be constructed of a bioerodible polymer, and can therefore dissolve one to six weeks after implantation into the body of the subject. Such bioerodible polymers can include, for example, polydioxanone, polylactic acid, and polyglycolic acid. Other bioerodible polymers are described in detail below.

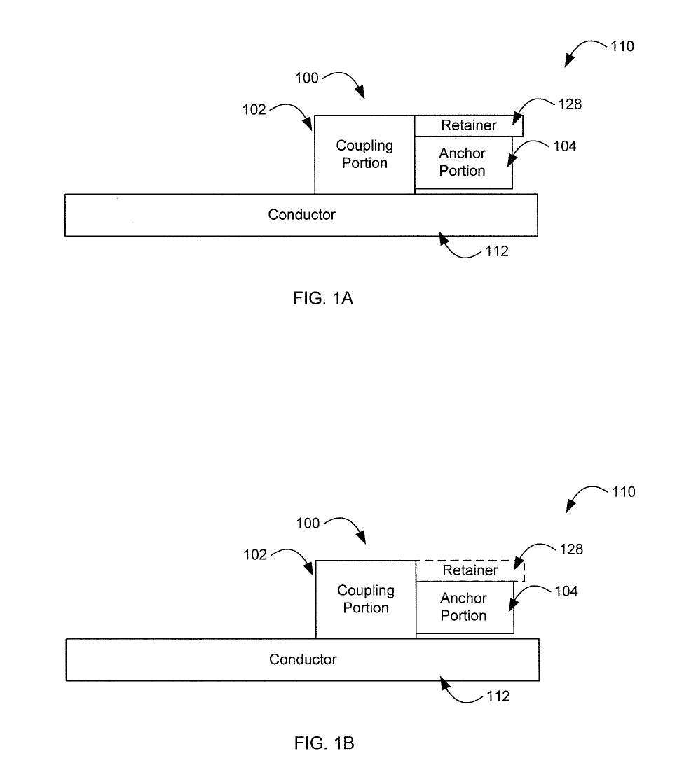

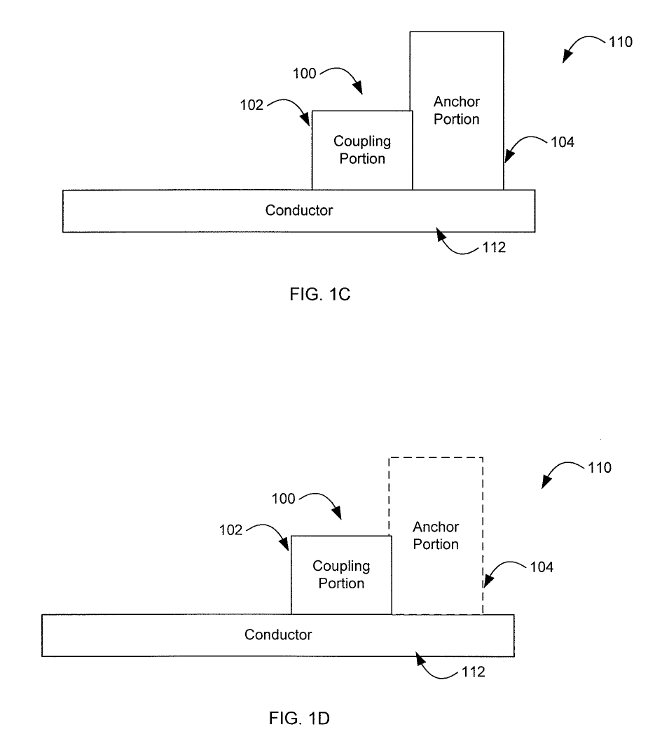

FIGS. 1A-1D are schematic illustrations of an implant 110, according to an embodiment of the invention disposed in a first configuration, a second configuration, a third configuration, and a fourth configuration, respectively. The implant 110 (also referred to herein as a “medical device”) can be placed or otherwise inserted into a body of a patient. The implant 110 includes a conductor 112 (also referred to herein as an “electrical conductor”), an anchor 100 (also referred to herein as a “tissue anchor”) and a retainer 128. The anchor 100 includes a coupling portion 102 and an anchor portion 104 (also referred to herein as a “retention portion”) adjacent the coupling portion 102. In some embodiments, the implant includes an insulating member (not shown) disposed about the conductor 112. The coupling portion 102 of the anchor 100 is coupled to the electrical conductor 112. The retainer 128 is coupled to the anchor portion 104 of the anchor 100.

The conductor 112 is configured to electrically stimulate target body tissue (not shown). Specifically, an electrical current can travel between the ends (or segments) of the conductor 112 to stimulate a target location such as muscle, a nerve or the like. The conductor 112 can be, for example, an electronic stimulator as described in U.S. patent application Ser. No. 12/187,655, entitled “Apparatus and Methods for Removing an Implant from a Body,” filed on Aug. 7, 2008, which is incorporated herein by reference in its entirety.

The coupling portion 102 of the anchor 100 can be coupled to the conductor 112 by any known coupling means capable of maintaining the coupling portion 102 of the anchor 100 in a fixed position relative to the conductor 112. Specifically, the coupling portion 102 of the anchor 100 can be coupled to the conductor 112 via, for example, an adhesive, an interference fit, a weld, a clip, etc.

The anchor portion 104 of the anchor 100 can move from a collapsed configuration to an expanded configuration. The anchor portion 104 is configured to permit movement of the implant 110 with respect to body tissue when the anchor portion 104 is in its collapsed configuration (as shown in FIG. 1A). The anchor portion 104 is configured to inhibit movement of the implant 110 with respect to body tissue (e.g., anchor the implant 110) when the anchor portion 104 is in its expanded configuration (as shown in FIG. 1C). Said another way, the anchor portion 104 limits movement of the implant 110 within a body when in its expanded configuration. Specifically, the anchor portion 104 can limit translational and/or rotational movement of the implant 110 when the anchor portion 104 is in its expanded configuration.

The anchor portion 104 of the anchor 100 can limit movement of the implant 110 when in its expanded configuration through any known anchoring means. For example, in some embodiments, the anchor portion 104 can include tines configured to engage body tissue such that anchor 100 is maintained in a substantially fixed position relative to the body tissue. In one or more of such embodiments, the tines can limit motion of the implant 110 in one direction (e.g., a proximal direction) while allowing motion in another direction (e.g., a distal direction). In other embodiments, the anchoring portion 104 can be a balloon configured to expand such that the balloon engages the body tissue and retains the anchor 100 in the position relative to the body tissue. In yet other embodiments, the retention portion 104 can be a screw, a clip, a hook, etc. In some embodiments, the retention portion 104 can include one or more retention mechanisms. For example, the retention portion 104 can include both tines and a balloon.

The anchoring portion 104 of the anchor 100 and the retainer 128 are bioerodible at a first rate and at a second rate, respectively. In other words, the anchor portion 104 erodes at the first rate when the anchor portion 104 is disposed within and/or contacts body tissue of the patient. Similarly, the retainer 128 erodes at the second rate when the retainer 128 is disposed within and/or contacts the body tissue of the patient. The second rate is greater than the first rate. Said differently, the retainer 128 substantially completely bioerodes before the anchor portion 104 substantially completely bioerodes. In some embodiments, the anchor portion 104 and/or the retainer 128 can release a biocompatible agent as they bioerode.

As shown in FIG. 1A, the retainer 128 retains the anchor portion 104 of the anchor in its collapsed configuration when the implant 110 is its first configuration. Said differently, the retainer 128 inhibits movement of the anchor portion 104 from its collapsed configuration to its expanded configuration. As shown in FIG. 1B, the retainer 128 substantially bioerodes when the implant 110 is disposed within body tissue for a first time period. The retainer 128 is shown as a dashed line in FIG. 1B to indicate that the retainer 128 has been eroded, absorbed and/or dissolved within the body. In some embodiments, the retainer 128 dissolves after the implant 110 is disposed within the body for a first time period such that it no longer retains the anchor portion 104 in its collapsed configuration when the implant 110 is in its second configuration (e.g., as shown in FIG. 1B).

After the retainer 128 is bioeroded, the anchor portion 104 moves from its collapsed configuration (as shown in FIG. 1B) to its expanded (deployed) configuration (as shown in FIG. 1C). In some embodiments, the anchor portion 104 of the anchor 100 is biased in its expanded configuration, thus the anchor portion 104 moves from its collapsed configuration to its expanded configuration without an external force. In other embodiments, an external force, signal and/or command can be applied (e.g., by a mechanical actuator, a hydraulic actuator, a pneumatic actuator, an electrical current or the like) to move the anchor portion 104 from its collapsed configuration to its expanded configuration. When the anchor portion 104 is in its expanded configuration, the anchor portion 104 can engage body tissue to retain the implant 110 with respect to the body tissue. Specifically, the implant 110 is anchored to body tissue when the implant 110 is in its third configuration (FIG. 1C).

As shown in FIG. 1D, the anchor portion 104 substantially bioerodes when the implant is disposed within body tissue for a second time period greater than the first time period. In other words, the anchor portion 104 dissolves such that it no longer anchors the implant 110 with respect to body tissue. The anchor portion 104 is shown as a dashed line in FIG. 1D to indicate that the anchor portion 104 has been eroded, absorbed and/or dissolved within the body. Thus, the implant 110 can move with respect to body tissue when the implant 110 is in its fourth configuration.

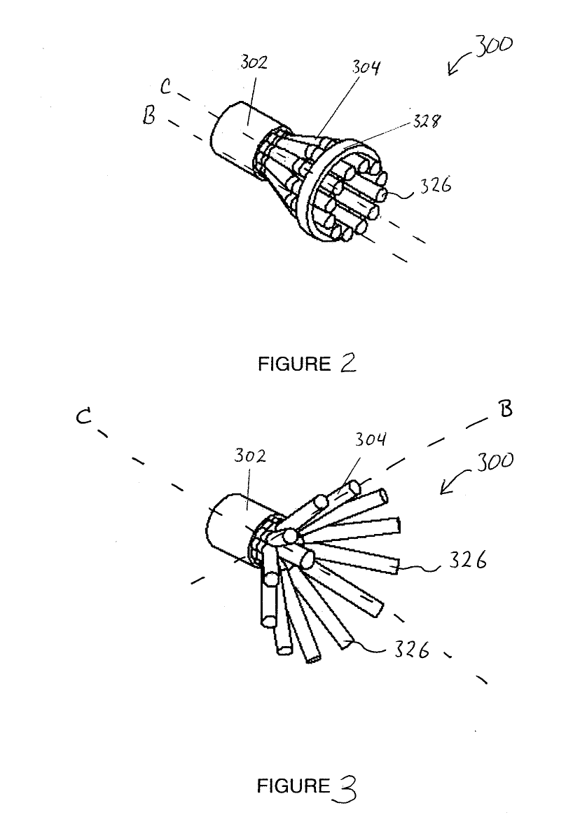

FIGS. 2 and 3 are perspective views of a tissue anchor 300 according to an embodiment of the invention in a first position (e.g., the collapsed configuration) and a second position (e.g., the expanded configuration), respectively. The tissue anchor 300 includes a coupling portion 302 and a retention portion 304 adjacent the coupling portion 302.

The coupling portion 302 is configured to be coupled to an electrical conductor (not shown). The electrical conductor can be any suitable electrical conductor of the types shown and described herein. The coupling portion 302 can be coupled to the electrical conductor by any suitable mechanism. For example, in some embodiments, the coupling portion 302 can define a lumen (not shown) configured to receive at least a portion of the electrical conductor. In such embodiments, the coupling portion 302 can be coupled to the electrical conductor via an interference fit. In other embodiments, the coupling portion 302 can be coupled to the electrical conductor via one of an interference fit, a weld, a threaded coupling and/or an adhesive such that the electrical conductor is maintained in a position relative to the tissue anchor 300. Said another way, the coupling portion 302 can be coupled to the electrical conductor such that movement (e.g., translational and/or rotational movement) of the electrical conductor with respect to the tissue anchor 300 is limited.

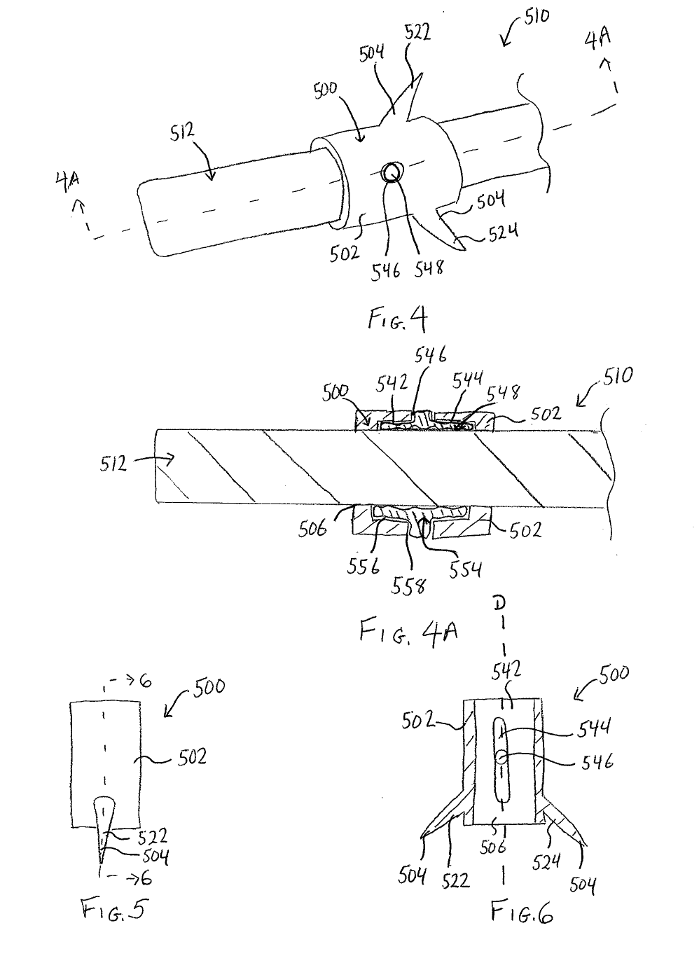

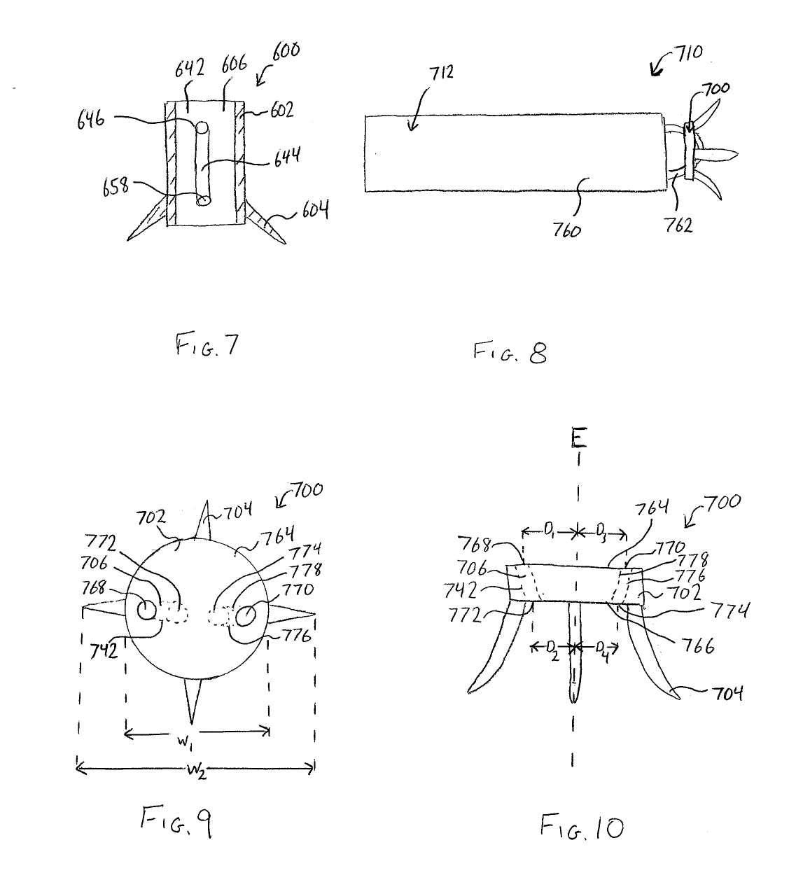

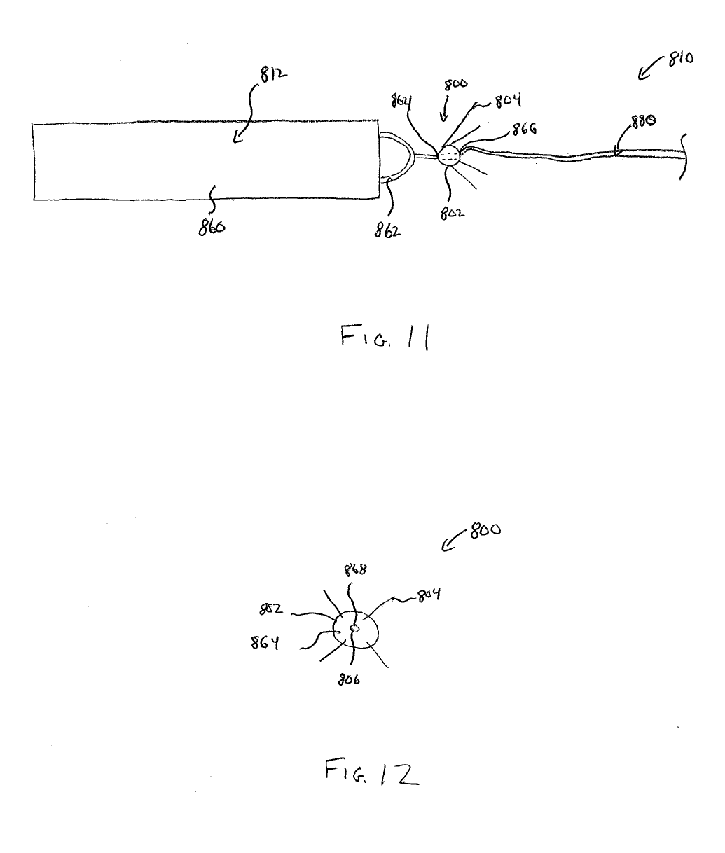

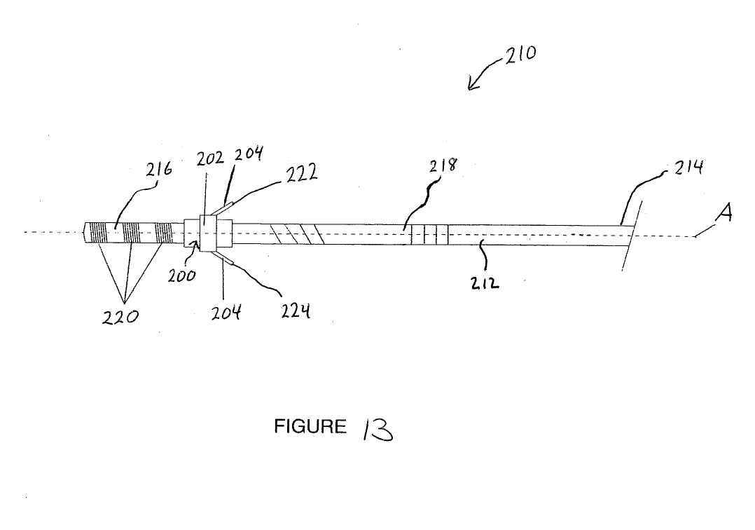

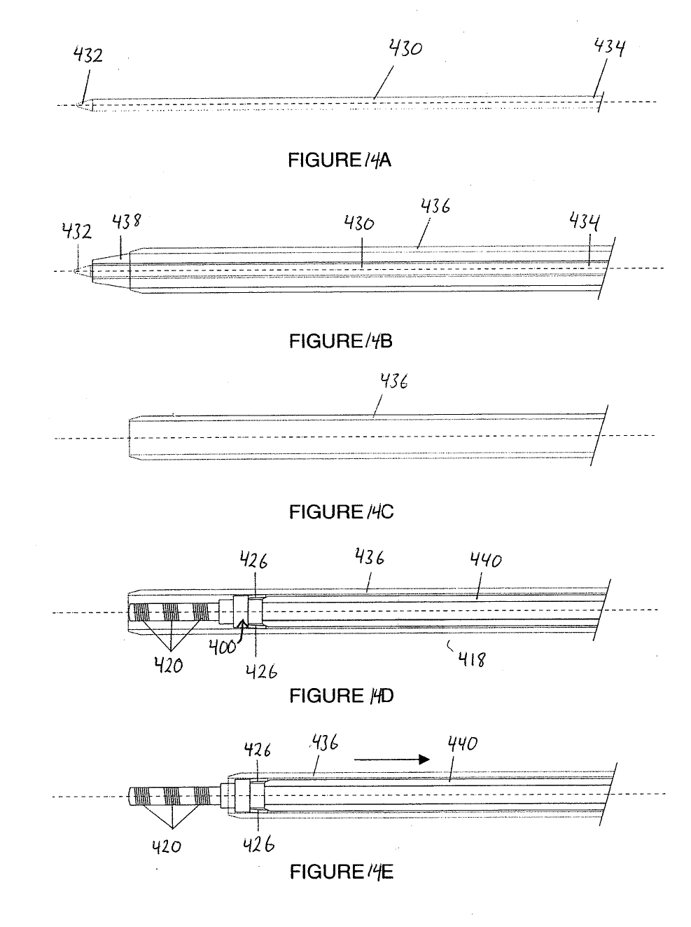

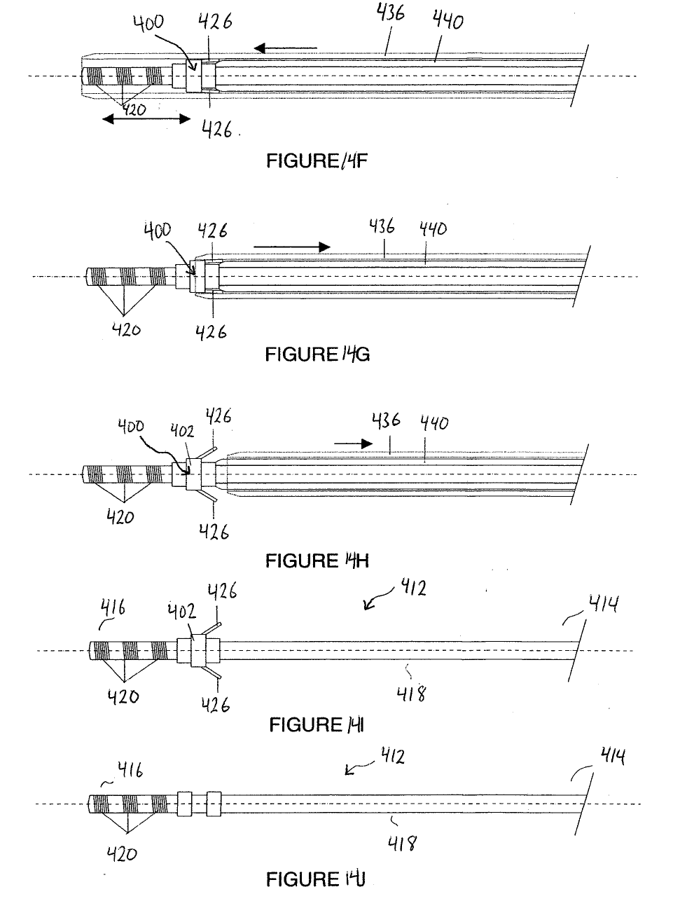

The retention portion 304 includes multiple tines 326 that are movable with respect to the coupling portion 302 of the tissue anchor 300. Thus, the retention portion 304 is movable between a collapsed configuration (FIG. 3) and an expanded configuration (FIG. 4). In this embodiment, the retention portion 304 is biased in the expanded configuration. Similarly stated, the retention portion 304 is nominally disposed in the expanded configuration. Accordingly, the retention portion 304 is constrained in the collapsed configuration by at least one anchor band or anchor strap 328 (also referred to herein as a “coupler”) configured to be disposed around the retention portion 304 and the electrical conductor, as illustrated in FIG. 2. In other words, the coupler 328 is coupled to the retention portion 304 and configured to inhibit movement of the retention portion 304 from the collapsed configuration to the biased expanded configuration.

When the tissue anchor 300 and/or the retention portion 304 are in the collapsed configuration, a portion of the electrical conductor including the tissue anchor 300 can be disposed within the body (not shown). Similarly stated, the retention portion 304 of the tissue anchor 300 can be held in its collapsed configuration via the coupler 328, for example, to facilitate insertion of the tissue anchor 300 into the body of the patient. Specifically, the tissue anchor 300 has a smaller profile (e.g., size) when the bioerodible retention portion 304 is in the collapsed configuration than when the bioerodible retention portion 304 is in the expanded configuration. More specifically, when the retention portion 304 is in the collapsed configuration a longitudinal axis B defined by at least one of the tines 326 of the retention portion 304 is substantially parallel to a longitudinal axis C defined by the tissue anchor 300, as shown in FIG. 2. Thus, when the tissue anchor 300 and/or the retention portion 304 are in the collapsed configuration, the portion of the electrical conductor can be moved within the body without the tines 326 substantially limiting movement of the electrical conductor. Similarly stated, when the tissue anchor 300 and/or the retention portion 304 are in the collapsed configuration, the portion of the electrical conductor can be moved within the body longitudinally (including both distally and proximally) and/or rotationally.

When the tissue anchor 300 and/or the retention portion 304 are in the expanded configuration, movement of the portion of the electrical conductor within the body is limited. More specifically, when the retention portion 304 is in the expanded configuration the longitudinal axis B defined by at least one of the tines 326 of the retention portion 304 is non-parallel to the longitudinal axis C defined by the tissue anchor 300, as shown in FIG. 3. More particularly, when the tissue anchor 300 and/or the retention portion 304 are in the expanded configuration, the tines 326 can engage and/or contact bodily tissue thereby limiting movement (e.g., regression) of the electrical conductor.

The retention portion 304 and the coupler 328 are constructed from a biocompatible material. The retention portion 304 and the coupler 328 are further configured to bioerode within the body of the patient. For example, the bioerodible retention portion 304 and the bioerodible coupler 328 are constructed of at least one bioerodible polymer, as discussed in detail below. Specifically, the bioerodible coupler 328 is formulated to bioerodible at a first rate and the retention portion 304 is formulated to bioerode a second rate that is slower than the first rate. Thus, after insertion into the body, the bioerodible coupler 328 bioerodes, thereby releasing the retention portion 304 (i.e., allowing the retention portion 304 to move to expanded configuration). Said another way, the bioerodible coupler 328 is coupled to the bioerodible retention portion 304 and is configured to maintain the bioerodible retention portion 304 in the collapsed configuration until the bioerodible coupler 328 substantially bioerodes.

In some embodiments, the coupler 328 is a silicone band. In some embodiments, the coupler 328 is formulated to substantially fully bioerode after being disposed within the body for less than 30 minutes. In this manner, a user can insert the electrical conductor and/or reposition the electrical conductor within the body when the retention portion 304 is maintained in the collapsed configuration before the coupler 328 allows the retention portion 304 to move to its expanded configuration. The coupler 328 is formulated to substantially fully bioerode after being disposed within the body for less than 30 minutes. In other embodiments, the coupler 328 is formulated to substantially fully bioerode after being disposed within the body for a period of time between 5 minutes and 20 minutes. In yet other embodiments, the coupler 328 is formulated to substantially fully bioerode after being disposed within the body for a period of time less than 1 hour.

The retention portion 304 and/or the tines 326 are configured to bioerode within the body of the patient. As described above, the tines 326 are bioerodible at a second rate that is slower than the first rate at which the bioerodible coupler 328 erodes. Thus, the retention portion 304 and/or the tines 326 can maintain the position of the electrical conductor within the body until the retention portion 304 and/or the tines 326 erode within the body. In some embodiments, for example, the retention portion 304 and/or the tines 326 can be configured to substantially fully bioerode after a time period during which the electrical conductor will be encapsulated within the body. In some embodiments, for example, the retention portion 304 and/or the tines 326 are formulated to substantially fully bioerode after being disposed within the body for a period of time of at least 14 days. In some embodiments, for example, the retention portion 304 and/or the tines 326 are formulated to substantially fully bioerode after being disposed within the body for a period of time of at least 21 days. In some embodiments, for example, the retention portion 304 and/or the tines 326 are formulated to substantially fully bioerode after being disposed within the body for a period of time between approximately 8 days and 14 days. In some embodiments, for example, the retention portion 304 and/or the tines 326 are formulated to substantially fully bioerode after being disposed within the body for a period of time between approximately 14 days and 21 days.

In some embodiments, each of the tines 326 are bioerodible at different rates. In some embodiments, the bioerodible retention portion 304 and the bioerodible coupler 328 are bioerodible at substantially the same rate. In such embodiments, the bioerodible coupler 328 has at least one of a width smaller than a width of each tine 326 and/or a surface area larger than a surface area of each tine 326 such that the bioerodible coupler 328 substantially bioerodes before the tines 326 substantially bioerode.

In some embodiments, at least one of the tines 326 and/or the coupler 328 is constructed of at least one biodegradable or bioerodible polymer that includes at least one biocompatible agent. The biocompatible agent can include, for example, a therapeutic agent (e.g., a medicament, a growth enhancing substance, or the like), conductive material, and/or insulative material. In this manner, the biocompatible agent is configured to be released into the body of the patient as the polymer bioerodes. For example, in some embodiments, the tines 326 and/or the coupler 328 can include a bioerodable coating that includes an agent. In other embodiments, the bioerodible polymer from which the tines 326 or the coupler 328 is constructed includes at least one biocompatible agent within the matrices of the polymer. The bioerodible polymer configured to include a therapeutic agent can be any suitable polymer, including, for example, polylactic acid, polyanhydride, polycaprolactone, and polyglycolic acid. The biocompatible agent, the release mechanisms (and/or kinetics) of the biocompatible agent, and the method of formulating the tines 326 and/or the coupler 328 can of the types shown and described in U.S. Patent Application Attorney Docket No. BION-005/01US 307799-2090, entitled “Electrical Conductor Having a Bioerodable Coating,” filed on Oct. 14, 2008, which is incorporated herein by reference in its entirety.

In some embodiments, the biocompatible agent is configured to be controllably released from at least one of the tines or the coupler into the body of the subject. For example, the biocompatible agent is configured to be controllably released by at least one of polymer erosion, diffusion, dispersion, osmosis, polymer swelling, or chemical control. With the exception of a swelling-controlled release system, the biocompatible agent can be configured to be eluted or released into the body of the subject in accordance with release kinetics based on laws of dispersion or Fickian diffusion.

In some embodiments, the biocompatible agent can be embedded in a reservoir or membrane system (not shown in FIGS. 2 and 3) of the tines 326 and/or the coupler 328. For example, in one embodiment, the polymer of the tissue anchor or the coupler includes a reservoir system configured to release the biocompatible agent. In other embodiments, the biocompatible agent is embedded in a matrix system. For example, the polymer of the tissue anchor and/or coupler includes a matrix system that is modeled based on porosity (or number of open pores), the nature of the loading mechanism as dissolved or dispersed, and the solubility limits in water.

FIG. 4 is a perspective view of a medical device 510 including an electrical conductor 512 coupled to a tissue anchor 500, according to an embodiment of the invention. FIG. 4A is a cross-sectional view of the medical device 510 of FIG. 4 along line 4A-4A. FIG. 5 is a side view of the tissue anchor 500 of FIG. 4. FIG. 6 is a cross-sectional view of the tissue anchor 500 of FIG. 5 along line 6-6. The tissue anchor 500 includes a coupling portion 502 and a retention portion 504 adjacent the coupling portion 502.

The retention portion 504 is configured to anchor the tissue anchor 500 and the electrical conductor 512 with respect to body tissue. Said another way, the retention portion 504 is configured to limit movement of 512 within the body. Specifically, the retention portion 504 includes a first tine 522 and a second tine 524 that each extend away from the electrical conductor 512. In this manner, when the medical device 510 is disposed within a body, the first tine 522 and the second tine 524 are configured to engage body tissue. Although in this embodiment, the retention portion 504 are tines 522 and 524, in other embodiments, the retention portion 504 can be any of a variety of different fixation/anchoring mechanisms, including for example, a screw, a clamp, etc.

The coupling portion 502 is configured to be coupled to the electrical conductor 512. The coupling portion 502 has an inner wall 542 defining a lumen 506 therethrough. The lumen 506 is configured to receive at least a portion of the electrical conductor 512. In other words, the coupling portion 502 includes a sleeve that can receive the electrical conductor 512. In the illustrated embodiment, the coupling portion 502 is coupled to the electrical conductor via a first adhesive 548 and a second adhesive 554. In alternative embodiments, the coupling portion is coupled to the electrical conductor via a weld and/or an interference fit. The tissue anchor 500 can have a length at or near 0.125 in. (0.3 cm). The coupling portion 502 of the tissue anchor 500 can have a width (e.g., an outer diameter) at or near 0.100 in. (0.3 cm). The lumen 506 of the tissue anchor 500 can have a width (e.g., an inner diameter) at or near 0.060 in. (0.2 cm).

The inner wall 542 of the coupling portion 502 further defines a first recess 544, a first aperture 546, a second recess 556, and a second aperture 558. The first recess 544 is in fluid communication with the lumen 506 and the first aperture 546. Similarly, the second recess 556 is in fluid communication with the lumen 506 and the second aperture 558. The first recess 544 is separate from the second recess 556. Said another way, the first recess 554 is fluidically isolated from the second recess 556 when the electrical conductor 512 is disposed within the lumen 506. The first recess 546 receives a portion of the first adhesive 548 therein when the first adhesive 548 is inserted and/or conveyed into the first aperture 546. The first adhesive 548 is configured to maintain at least the coupling portion 502 of the tissue anchor 500 in a fixed position relative to the electrical conductor 512. Said another way, the first adhesive 548 prevents migration of the electrical conductor 512 relative to the tissue anchor 500. In the illustrated embodiment, a portion of the first adhesive 548 is disposed between the electrical conductor 512 and a portion 550 of the inner wall 542 of the coupling portion 502 defining the first recess 544. The first adhesive 548 is also disposed between a portion 552 of the inner wall 542 of the coupling portion 502 defining the first aperture 546. In some embodiments, the inner wall 542 only defines the first aperture (i.e., the first aperture 546 and the first recess 544 share a common boundary). In some embodiments, a portion of the first adhesive is disposed between the electrical conductor and the inner wall of the coupling portion defining the lumen. In some embodiments, a portion of the first adhesive is disposed within the first aperture such that the first adhesive inhibits fluid communication between the first recess and the outer surface of the tissue anchor.

Similarly, the inner wall 542 of the coupling portion 502 defines a second recess 556 and a second aperture 558. As described above, the second recess 556 is separate from the first recess 544. The second recess 556 is in fluid communication with the lumen 506 and the second aperture 558. The second recess 556 receives the second adhesive 554 therein when the second adhesive 554 is inserted into the second aperture 558. The second adhesive 554 is configured to maintain at least the coupling portion 502 of the tissue anchor 500 in a position relative to the electrical conductor. In other words, the second adhesive 554 prevents migration of the electrical conductor 512 relative to the tissue anchor 500. In some embodiments, the inner wall can define more or less than two recesses/apertures configured to receive adhesive therein.

In the illustrated embodiment, the first recess 544, as shown in FIG. 6, and the second recess 556 each extend along an axis D defined by the inner wall 542 of the coupling portion 502 defining the lumen 506. In other embodiments, the first recess 544 and/or the second recess 556 can extend along a circumference of the inner wall of the coupling portion defining the lumen. In yet other embodiments, the first recess 544 and/or the second recess 556 extends along the axis defined by the inner wall of the coupling portion defining the lumen and along the circumference of the inner wall of the coupling portion defining the lumen (e.g., helically, spirally, etc.).

Although in this embodiment, adhesives 548 and 554 maintain the position of the electrical conductor 512 in a fixed position relative to the tissue anchor 500, it should be understood that other mechanisms can be used to limit movement of the tissue anchor 500 relative to the electrical conductor 512. For example, such mechanisms can include a clamp, a bolt and screw, a band, weld, solder joint or the like.

As described above, the first recess 544 is separate from the second recess 556. In this manner, the first adhesive 548 can be different from the second adhesive 554. Using two different adhesives can, for example, improve the strength of the bond between the electrical conductor 512 and the tissue anchor 500.

FIG. 7 is a cross-sectional view of a tissue anchor 600 according to an embodiment of the invention. The tissue anchor 600 includes a coupling portion 602 and a retention portion 604. The coupling portion 602 includes an inner wall 642 defining a lumen 606 and a recess 644 in fluid communication with the lumen 606. In this embodiment, the inner wall 642 of the coupling portion 602 defines a first aperture 646 and a second aperture 658 separate from the first aperture 646. Similarly stated, the first aperture 646 and the second aperture 658 do not share a common boundary (i.e., non-continuous). The first aperture 646 and the second aperture 658 are each in fluid communication with the recess 644. The recess 644 is configured to receive an adhesive (not shown) therein when the adhesive is inserted into at least one of the first aperture 646 and the second aperture 658. In some embodiments, more or less than two apertures can be in communication with the recess such that the adhesive is received in the recess when the adhesive is inserted into any aperture in communication with the recess. In this manner, the adhesive is more easily inserted into the recess and more evenly distributed within the recess. For example, this arrangement can allow an adhesive to be conveyed into the recess 644 via the first aperture 646 while air can be conveyed from the recess 644 via the second aperture 658.

FIG. 8 is a side view of a medical device 710 including an electrical conductor 712 coupled to a tissue anchor 700, according to an embodiment of the invention. FIGS. 9 and 10 are a front view and a top view, respectively, showing internal components of the tissue anchor 700 of FIG. 8. The electrical conductor 712 includes a conductor portion 760 and an eyelet portion 762. The conductor portion 760 is configured to stimulate target body tissue (not shown) of a patient. The eyelet portion 762 is configured to be coupled to a suture (not shown). Although the medical device 710 is shown and described as including an electrical conductor 712, in other embodiments, the medical device 710 can include any elongated device and/or implantable device.

The tissue anchor 700 includes a coupling portion 702 and a retention portion 704. The retention portion 704 is configured to anchor the electrical conductor 712 with respect to body tissue as described herein. The coupling portion 702 has a first diameter W1 (e.g., a first width). The retention portion 704 has a second diameter W2 (e.g., a second width) greater than the first diameter W1, as shown in FIG. 9.

The tissue anchor 700 has a first end surface 764 and a second end surface 766 opposite the first end surface 764. The first end surface 764 of the tissue anchor 700 defines a first opening 768 and a second opening 770 separate from the first opening 768. The second end surface 766 of the tissue anchor 700 defines a third opening 772 and a fourth opening 774 separate from the third opening 772. The tissue anchor 700 has a first inner wall 742 that defines a first lumen 706 extending from the first opening 768 to the third opening 772. The tissue anchor 700 has a second inner wall 776 that defines a second lumen 778 extending from the second opening 770 to the fourth opening 774.

The first opening 768 and an axis E defined by the tissue anchor 700 is separated by a first distance D1. The third opening 772 and the axis E defined by the tissue anchor 700 is separated by a second distance D2. The second distance D2 is less than the first distance D1. In other words, the centerline of the first lumen 706 is non-parallel with the axis E. Similarly, the second opening 770 and the axis E defined by the tissue anchor 700 is separated by a third distance D3. The fourth opening 774 and the axis E defined by the tissue anchor 700 is separated by a fourth distance D4. The fourth distance D4 is less than the third distance D3. In the illustrated embodiment, the first distance D1 is substantially equal to the third distance D3. Similarly, the second distance D2 is substantially equal to the fourth distance D4. Similarly stated, the lumens 706 and 778 and the openings 768, 770, 772 and 774 are substantially symmetrical about the axis E, respectively. In an alternative embodiment, the first distance is different than the third distance. Similarly, the second distance is different than the fourth distance.

The coupling portion 702 of the tissue anchor 700 is configured to be coupled to the eyelet portion 762 of the electrical conductor 712. Specifically, the eyelet portion 762 of the tissue anchor 700 is disposed within the first lumen 706 and the second lumen 778 when the tissue anchor 700 is coupled to the eyelet portion 762. Thus, the first inner wall 742 and the second inner wall 776 engage the eyelet portion 762 of the electrical conductor 712 such that a movement in a direction by the electrical conductor 712 results in a movement of the tissue anchor 700 in that direction. Thus, after the tissue anchor 700 becomes anchored to body tissue, the tissue anchor 700 prevents migration of the electrical conductor 712 with respect to that body tissue. Said another way, movement of the tissue anchor 700 with respect to the electrical conductor 712 is limited. In the illustrated embodiment, the tissue anchor 700 is directly coupled to the eyelet portion 762 of the electrical conductor 712. In an alternative embodiment, the tissue anchor is indirectly coupled to the eyelet portion of the electrical conductor.

In the illustrated embodiment, at least one end of the eyelet portion 762 is removably coupled to the electrical conductor such that the tissue anchor 700 can be coupled to the eyelet portion 762 via threading the eyelet portion 762 through the first lumen 706 and through the second lumen 778.

FIG. 11 is a side view of a medical device 810 including an electrical conductor 812 coupled to a tissue anchor 800 according to an embodiment of the invention. FIG. 12 is a front view of the tissue anchor 800 of FIG. 11. The electrical conductor 812 includes a conductor portion 860 and an eyelet portion 862. The tissue anchor 800 includes a coupling portion 802 (e.g., a body portion) and a retention portion 804. In this embodiment, the medical device 810 includes a suture 880 coupled to the eyelet portion 862 of the electrical conductor 812.

The coupling portion 802 of the tissue anchor 800 is configured to be coupled to the suture 880. Specifically, the tissue anchor 800 has a first end portion 864 and a second end portion 866 opposite the first end portion 864. The first end portion 864 of the tissue anchor 800 defines a first opening 868. The second end portion 866 of the tissue anchor 800 defines a second opening (not shown). The tissue anchor 800 defines a lumen 806 extending from the first opening 868 to the second opening. The lumen 806 is configured to receive the suture 880. In this embodiment, the tissue anchor 800 is coupled to the suture 880 via an interference fit. In some embodiments, the tissue anchor 800 is coupled to the suture via a weld, an adhesive or the like to maintain the tissue anchor 800 in a position with respect to at least a portion of the suture.

Although in this embodiment, the coupling portion 802 of the tissue anchor 800 is substantially spherical in shape, it should be understood that the coupling portion 802 of the tissue anchor 800 can be any of a variety of different shapes. For example, the coupling portion can be substantially ovular in shape, cylindrical in shape, conical in shape, rectangular in shape, etc.

In some embodiments, the anchor 800, the suture 880, and/or the eyelet portion 862 can be bioerodible and/or dissolvable, as described herein.

FIG. 13 illustrates a medical device 210, according to an embodiment of the invention. The medical device 210 is configured to provide a conductive pathway for at least a portion of an electrical current originating from a stimulator (not shown) to a target body tissue (not shown) in a body of a subject. For example, the target body tissue can include subcutaneous tissue, a neural tissue (i.e., in the peripheral or central nervous system), a nerve, a muscle (e.g., skeletal, respiratory, or cardiac muscle), an organ (e.g., the brain, cochlea, optic nerve, heart, bladder, urethra, or kidney), or bone. The medical device 210 includes an electrical conductor 212 and a tissue anchor 200.

The electrical conductor 212 includes a pick-up end 214, a stimulating end 216 and a lead portion 218 extending between the pick-up end 214 and the stimulating end 216. The pick-up end 214 of the electrical conductor 212 is configured to be electrically coupled or connected to a stimulator (not shown). In some embodiments, the pick-up end 214 of the electrical conductor 212 is coupled to an electrode-battery assembly (not shown) that is coupled to the stimulator. The pick-up end 214 is configured to receive an electrical current transmitted from the stimulator. The pick-up end 214 is configured to transmit the electrical current to the lead portion 218 of the electrical conductor 212.

The lead portion 218 is configured to transmit the electrical current from the pick-up end 214 to the stimulating end 216 of the electrical conductor 212. In some embodiments, the lead portion 218 of the electrical conductor 212 is configured to be inserted percutaneously into the body such that the stimulating end 216 (e.g., the distal end portion) is adjacent to a target body tissue.

The stimulating end 216 of the electrical conductor 212 is configured to transmit at least a portion of the electrical current from the electrical conductor 212 to the target body tissue. In this manner, the electrical current can stimulate the target body tissue by at least partially activating or blocking the conduction or propagation of action potentials or nerve impulses along the axons of nerves at or near the target body tissue. More particularly, the stimulating end 216 of the electrical conductor 212 transmits the electrical current to the target body tissue via at least one conductive stimulating electrode 220. In the illustrated embodiment, the stimulating end 216 of the electrical conductor 212 includes three conductive stimulating electrodes 220.

The tissue anchor 200 includes a coupling portion 202 and a retention portion 204 adjacent to the coupling portion 202. The coupling portion 202 of the tissue anchor 200 is coupled to the electrical conductor 212. Specifically, the coupling portion 202 is configured to be disposed over or on at least a portion of the electrical conductor 212. More specifically, the coupling portion 202 is disposed over or on a portion of the lead portion 218 of the electrical conductor 212. In another embodiment, the coupling portion 202 is disposed over or on a portion of the stimulating end 216 of the electrical conductor 212 or the pick-up end 214 of the electrical conductor 212.

The retention portion 204 is coupled to the electrical conductor 212 by the coupling portion 202 of the tissue anchor 200. In one embodiment, no cavity develops in the coupling portion 202 when the retention portion 204 bioerodes.

The retention portion 204 of the tissue anchor 200 is configured to anchor the electrical conductor 212 to within the body of the patient. Specifically, the retention portion 204 is configured to limit a backward (or proximal) movement of the electrical conductor 212 during insertion of the electrical conductor 212 into the body and/or after the electrical conductor 212 is disposed within the body. In some embodiments, the retention portion 204 can be configured to prevent forward (or distal) movement and/or rotational movement of the electrical conductor after the electrical conductor 212 is disposed within the body.

The retention portion 204 is configured to move from a first position (e.g., a collapsed configuration not shown in FIG. 13) to a second position (e.g., an expanded configuration as shown in FIG. 13). The retention portion 204 is configured to facilitate insertion of the electrical conductor 212 when the retention portion 204 is in the collapsed configuration. The retention portion 204 is substantially parallel with the lead portion 218 of the electrical conductor 212 when the retention portion 204 is in its collapsed configuration. The retention portion 204 extends outwardly from the electrical conductor 212 when the retention portion 204 is in its expanded configuration. In this manner, the retention portion 204 can engage body tissue of the patient when the retention portion 204 is in the expanded configuration.

Although the electrical conductor 212 is illustrated and described as including one tissue anchor 200 disposed about one portion of the electrical conductor 212, in another embodiment, the electrical conductor includes more than one tissue anchor. For example, in one embodiment, the electrical conductor includes a first tissue anchor disposed on or defined by the electrical conductor on a first portion of the electrical conductor and a second tissue anchor disposed on or defined by the electrical conductor on a second portion of the electrical conductor different than the first portion.

The retention portion 204 includes a first tine 222 and a second tine 224 configured to engage body tissue such that the tissue anchor 200 can be anchored to the body tissue. Although two tines 222 and 224 are illustrated in FIG. 13, it should be understood that the retention portion 204 can include any number of tines. The tines 222 and 224 are configured to extend outwardly from the electrical conductor 212 at an angle of at least 15 degrees from the longitudinal axis A defined by the electrical conductor 212 when the retention portion 204 is in the expanded configuration. In other embodiments, the tines 222 and 224 are configured to extend outwardly from the electrical conductor 212 at an angle up to 90 degrees from the longitudinal axis A defined by the electrical conductor 212 when the retention portion 204 is in the expanded configuration. Although the tines 222 and 224 are described herein as extending from the electrical conductor 212 at an angle of at least 15 degrees and an angle up to 90 degrees, in another embodiment, the tines 222 and 224 can extend from the electrical conductor 212 at any angle.

In some embodiments, the first tine can extend from the electrical conductor at an angle different than an angle at which the second tine extends. For example, in one embodiment, the first tine is configured extend from the electrical conductor at a 15 degree angle to the longitudinal axis A and the second tine is configured to extend from the electrical conductor at a 30 degree angle to the longitudinal axis A. In still another embodiment, a retention portion can be devoid of tines.

The tines 222 and 224 are configured to minimize tissue trauma that may occur when the electrical conductor 212 is explanted from the body of the subject. For example, in one embodiment, the tines 222 and 224 are constructed of a flexible or pliant material. In another embodiment, the fixation tines 222 and 224 are constructed of a bioerodible material. In such an embodiment, the electrical conductor 212 can be explanted when the tines 222 and 224 substantially bioerode. The bioerodible material can be flexible or rigid.

The size or dimensions of the tines 222 and 224 is configured to help minimize tissue trauma, such as when the electrical conductor 212 is explanted from the body of the patient. For example, in one embodiment, the tines define a width in the range of about 0.1 mm to about 1.0 mm. In another example, the tines define a thickness in the range of about 0.1 mm to about 0.5 mm. In another example, the tines may have a circular cross-section, varying in the diameter range from about 0. 1 mm to about 0.5 mm. In yet another example, the tines define a length in the range of about 1.0 mm to about 5.0 mm.

FIGS. 14A-14J illustrate an electrical conductor being inserted into a body of a patient (not shown), according to an embodiment. As illustrated in FIG. 14A, a probe 430 is inserted into body tissue of the patient. In one embodiment, the probe 430 is inserted into the body of the subject through the skin. The stimulating tip 432 of the probe 430 is positioned proximate to the target body tissue. For example, in one embodiment, the stimulating tip 432 is positioned proximate to a nerve (not shown).

A stimulation, such as an electrical current, is provided to the connecting end 434 of the probe 430. The stimulation is transmitted through the probe 430 to the stimulating tip 432. The response of the target body tissue to the stimulation is monitored or tested. A position for implanting the electrical conductor 412 is identified once the stimulating tip 432 is in a position sufficient to stimulate the target body tissue and generates a desired response.

Once the position for placement or implanting of the electrical conductor 412 is determined, a pathway is defined in the body tissue for insertion of the electrical conductor 412. As illustrated in FIG. 14B, a pathway can be defined by inserting a sheath 436 into the body of the patient over the probe 430. The sheath 436 is configured to receive the probe 430 and a dilator 438. The dilator 438 is inserted into the body of the patient between the probe 430 and the sheath 436.

The dilator 438 defines a length which extends from the stimulating tip 432 of the probe 430 to an indicating mark (not shown) on the probe 430. In one embodiment, the sheath 436 defines a length which corresponds to the length of the dilator 438. In one embodiment, the sheath 436 and dilator 438 are similar to a standard intravenous dilator set which latches together with a locking mechanism, for example, a Luer-lock.

The sheath 436 can be formed of a plastic material, for example Teflon™ FEP (DuPont). The dilator 438 is formed of plastic, for example, high density polyethylene or surgical grade stainless steel. The sheath 436 defines an inner diameter that is approximately equal to an outer diameter defined by the dilator 438. In one embodiment, the sheath defines a diameter of 2.3 mm, which is equivalent to a 7-French dilator set.

The dilator 438 dilates the pathway for insertion of the electrical conductor 412 in the body of the patient. As illustrated in FIG. 14C, the probe 430 and the dilator 438 are each withdrawn from the body of the subject. In one embodiment, saline is injected into the sheath 436 to increase the electrical conductivity into the tissue before insertion of the electrical conductor 412.

As illustrated in FIG. 14D, at least a portion of the electrical conductor 412 is inserted into a loader 440. For example, the lead portion 418 of the electrical conductor 412 is inserted into the loader 440. The loader 440 defines a length that extends at least from the pick-up end 414 of the electrical conductor 412 to the tines 426 of the tissue anchor 400. The loader 440 includes at least one position indicator. In one embodiment, the loader 440 includes three position indicators (not shown). A first position indicator is a first mark indicating an insertion depth. A second position indicator is a second mark indicating an exposed electrode. A third position indicator is a third mark indicating deployment of the tissue anchor 400.

The portion of the loader 440 in which the lead portion 418 of the electrical conductor 412 is inserted is inserted into the sheath 436. In one embodiment, the loader 440 is inserted into the sheath 436 to a depth indicated on the loader 440 by the first mark (not shown).

As illustrated in FIG. 14D, the tines 426 are constrained in an undeployed position by an inner wall of the sheath 436. In another embodiment, the tines 426 are constrained by at least one band (not shown) configured to be disposed around the tines in an undeployed position. The band is configured to constrain the tines 426 to a diameter less than or equal to the inner diameter of the sheath 436. The band can be any of the bands, anchor straps and/or couplers shown and described herein. For example, in some embodiments, the tines 426 can be maintained in the undeployed position by a band similar to the coupler 328 shown and described above.

As illustrated in FIG. 14E, the sheath 436 is at least partially withdrawn from the body of the subject. Said another way, the sheath 436 is withdrawn or moved in a proximal direction, as indicated by the arrow in FIG. 14E, from being disposed about the stimulating end 416 of the electrical conductor 412. In one embodiment, the sheath 436 is withdrawn to the second mark (not shown) on the loader 440 to expose the stimulating electrodes 420 to the target body tissue.

An electrical current is transmitted through the electrical conductor 412 to the stimulating electrodes 420 to test the response of the target body tissue. If the position of the electrical conductor 412 requires adjustment, the sheath 436 is re-inserted, or moved in the distal direction as indicated by the top arrow in FIG. 14F. In one embodiment, the loader 440 is held substantially stationary as the sheath 436 is moved in the proximal direction and/or in the distal direction.

If the position of the electrical conductor 412 requires adjustment, the assembly of the sheath 436, loader 440, and electrical conductor 412 is adjusted. In one embodiment, the assembly is adjusted by moving the assembly in a forward (or distal) direction. In one embodiment, the assembly is adjusted by moving the assembly in a backward (or proximal) direction. The assembly can be alternatively moved in the forward and backward directions, as indicated by the lower arrows in FIG. 14F, until the electrical conductor 412 is in the desired or most efficacious position. In another embodiment, minor adjustments are made to the position of the electrical conductor 412 without re-inserting the sheath 436.

After adjusting the position of the electrical conductor 412, the sheath is withdrawn, as indicated by the arrow in FIG. 14G to expose the stimulating electrodes 420 to the target body tissue. The response of the target body tissue to stimulation is re-tested. Adjustment of the electrical conductor 412 and re-testing of the target body tissue's response to stimulation can be repeated as needed.

Once the electrical conductor 412 is in the desired or most efficacious position, the sheath 436 is withdrawn to the third mark (not shown) on the loader 440. In some embodiments, withdrawing the sheath 436 to the third mark exposes the tines 426, as illustrated in FIG. 14H. With the sheath 436 withdrawn (or removed), the tines 426 move to the deployed position. The deployed tines 426 anchor the electrical conductor 412, and thus the stimulating electrodes 420, in the desired or most efficacious position for stimulating the target body tissue.

Each of the loader 440 and the sheath 436 are fully withdrawn from the electrical conductor 412. The electrical conductor 412 remains positioned within the body tissue of the subject, as illustrated in FIG. 14I.

As illustrated in FIG. 14J, in some embodiments, the tissue anchor 400 coupled to the electrical conductor 412 can include bioerodible tines 426, of the types shown and described herein. In this manner, when the tines 426 are in the deployed configuration (see FIG. 14I), the tines 426 can limit the movement of the electrical conductor 412 within the body. As shown in FIG. 14J, the tines 426 erode within the body after a time period. Thus, in some embodiments, the tines 426 anchor the electrical conductor 412 until partial or full encapsulation of the electrical conductor 412 by body tissue occurs. Encapsulation tissue forms around the electrical conductor 412 to fill the available space surrounding the implanted electrical conductor 412 and create a protective barrier against migration of the electrical conductor 412. Over time, the tines 426 bioerode (and/or dissolve) in the body tissue. Once the tines 426 have bioeroded, the electrical conductor 412 can be removed without causing tissue damage that can be associated with removal of a fixation mechanism including non-bioerodible tines. In another embodiment, as the tines 426 bioerode, at least one therapeutic agent is released into the body of the subject, as described herein.

In one embodiment, the passive electrical conductor 412 is entirely implanted within the body of the patient. In another embodiment, the passive electrical conductor is implanted percutaneously. For example, percutaneous stimulation can be used to deliver stimulation without activating the skin receptors. A percutaneous electrical conductor is used, for example, to deliver an electrical stimulation through the skin to a target body tissue to treat a condition, such as pain, or to activate a motor point. For example, the percutaneous electrical conductor delivers an electrical stimulation to activate a motor point by at least partially inducing the conduction or propagation of action potentials or nerve impulses along the axons of a target nerve associated with the motor point.

The electrical conductors shown and described herein can be constructed of any material suitable for transmitting or routing an electrical current within a body of a subject. For example, in some embodiments, the electrical conductor is constructed of at least one of a metal wire, carbon fibers, a conductive rubber or other conductive polymer, or a conductive salt solution in rubber. Some known conductors of the type used in cardiac pacemaker leads are constructed of multi-stranded, Teflon®-insulated, stainless-steel wire. In another embodiment, the electrical conductor is constructed of at least one of MP35N® alloy (a nonmagnetic, nickel-cobalt-chromium-molybdenum alloy) or alloys of platinum and/or iridium which are commonly used in parts for medical applications.

The polymers described herein (e.g., as included in the tines, the retention portions and/or couplers described herein) can be of any suitable formulation. For example, in some embodiments, at least one of the tines, the retention portion and/or the coupler is constructed of or includes a polymer selected from a class of plastics that adheres to the ISO 10993 standards for prolonged and permanent implantation in a body of a subject. In other embodiments, a polymer used in the construction of the tissue anchors described herein is a Class VI plastic as identified by the United States Pharmacopeia. In some embodiments, a solvent used in the preparation of the polymer from which the tissue anchors described herein are constructed can be selected from a group of solvents identified as acceptable according to ISO standards and the United States Pharmacopeia, such as a solvent that is at least a Class II or III solvent according to the United States Pharmacopeia.

The bioerodable polymers described herein (e.g., as included in the tines, the retention portions and/or couplers described herein) can be of any suitable formulation. For example, such bioerodible polymers can be selected from at least one of: poly[bis(p-carboxyphenoxy)propane anhydride] (pCPP):sebacic acid (SA), polylactic acid, polyanhydride, polycaprolactone, and polyglycolic acid. Bioerodible polymers are also described or discussed in U.S. Pat. No. 5,030,457 to Ng et al., U.S. Pat. Nos. 5,939,453 and 5,968,543 to Heller et al., U.S. Pat. No. 6,153,664 to Wise et al., and U.S. Pat. No. 6,304,786 to Heil Jr. et al., each of which are incorporated herein by reference. Other bioerodible polymers include polydioxanone; aliphatic or other polyesters including polycaprolactone and polyglycolide; modified polysaccharides and other natural polymers including cellulose acetate butyrate; poly(ethylene glycol) based polymers including poly(ethylene) oxide; poly(ethylene glycol)-poly(propylene glycol) based polymers including poly(ethylene glycol-ran-propylene glycol); poly(vinyl alcohol) and copolymers including poly(vinyl alcohol-co-ethylene); hydrogels or other crosslinked polymers including poly(N-isopropylacrylamide); hydrophilic polymers including polyvinylpyrrolidone; hydrophobic polymers including poly(4-vinylphenol); and/or an appropriate crosslinker including divinylbenzene.

The bioerodable polymers and/or the biocompatible agents described herein can be released into the body by any suitable mechanism. For example, in some embodiments, the bioerodable polymers and/or the biocompatible agent described herein can be controllably released into the body of the patient by polymer erosion. For example, in some embodiments, polymer erosion occurs when a hydrophilic polymer undergoes hydrolysis. Hydrolysis can occur by hydrolytic cleavage of the cross-links or backbone. In other embodiments, polymer erosion occurs when a water insoluble polymer is solubilized by hydrolysis, ionization, or pronation of a pendant group. In still another example, polymer erosion occurs when a hydrophobic polymer undergoes backbone cleavage resulting in a transformation to small water-soluble molecules.

In other embodiments, the bioerodable polymers (e.g., used in the construction of the retention portion and/or the coupler) and/or the biocompatible agents described herein can be released into the body by a dissolution-controlled system. In one embodiment, the dissolution-controlled system combines polymer swelling and slow macromolecular chain disentanglement, which cooperatively causes the controlled release of the biocompatible agent.

Various factors can trigger bioerosion including, for example, exposure to an aqueous substance, such as bodily fluids, changes in temperature or electrical stimulation, and changes in pH. For example, esterified copolymers of methyl vinyl ether and maleic anhydride have a pH threshold range of only 0.25 pH unit, therefore a very small degree of ionization can solubilize the polymer. The rate of drug (or other agent) release in these cases will follow a linear relationship with polymer erosion.

In some embodiments, a method includes producing a mathematical model of the elution, release, erosion and/or dissolution of the bioerodable polymers (e.g., used in the construction of the retention portion and/or the coupler) and/or the biocompatible agents described herein. For example, in some embodiments a method includes comparing the modeled elution, release, erosion and/or dissolution of the bioerodable polymers and/or the biocompatible agents to in vitro and in vivo functions. In this manner, the elution, release, erosion and/or dissolution of the bioerodable polymers and/or the biocompatible agent can be optimized. For example, in some embodiments, the composition ratios of polymer to polymer or biocompatible agent to polymer can be customized by a practitioner or manufacturer and modeled for specific applications depending on the strength and duration of the desired elution or release. In some embodiments, for example, a kit can include an electrical conductor (e.g., lead) and multiple tissue anchors. Each of the tissue anchors can be formulated to have different bioerosion properties. In this manner, a user can select the tissue anchor having the desired bioerosion properties and couple the selected tissue anchor to the electrical conductor prior to insertion of the electrical conductor.

In some embodiments, the therapeutic agent is configured to help treat or alleviate infection, inflammation, and/or pain, which may arise due to implantation of the electrical conductor. The therapeutic agent included, disposed, or incorporated in the polymer can include or be selected from, for example, antimicrobial agents, including polysporins; anti-inflammatory agents, including corticosteroids; and pain reducers, including dibucaine. Release of a known therapeutic agent, namely dibucaine free base, dibucaine HCl, and bupivacaine HCl, from a bioerodible polymer matrix.

In some embodiments, at least one of the tissue anchor or the coupler is or includes a bioerodible polymer configured to help recover electrical current lost and or limit the attenuation of current to surrounding body tissue. For example, at least one of the tissue anchor or the coupler includes or is constructed of at least one bioerodible polymer that includes at least one conductive agent or material, or is constructed of a bioerodible polymer that is conductive. For example, the bioerodible polymer can be electrically conductive or can be configured to deliver an electrically conductive material or agent to the surrounding body tissue as the polymer erodes (or degrades).

In some embodiment, the tissue anchor or the couplers described herein is or includes a bioerodible polymer that is more conductive than the body tissue surrounding the electrical conductor. For example, subcutaneous tissues have been shown to have conductivity of 0.04 S/m with a stimulation frequency less than 100 Hz. Thus in some embodiments, a tissue anchor or coupler can include a bioerodible polymer constructed of a complex of polyacid-poly(N-vinyl pyrrolidone), which has been shown to have conductivity greater than 0.04 S/m.

In some embodiments, at least one of the tissue anchors or the couplers described herein includes or is constructed of at least one bioerodible polymer that includes at least one insulative agent or material. For example, in one embodiment, the tines are configured to deliver an insulative medium, polymer, or particles to the surrounding body tissue as the polymer of which the tines are constructed bioerode. In some embodiments, the insulative agent is an electrically insulative polymer or particles. The insulative agent is configured to increase the transfer of electrical current to the target body tissue. Because electrical current may “escape” into surrounding tissues as the current is transferred along the electrical conductor, release of the insulative agent into the surrounding body tissue helps to prevent loss of (or helps recover) “escaping” electrical current.

Although the bioerodible polymers are described herein as being included in a tissue anchor and/or a coupler, in other embodiments, a different portion of the electrical conductor and/or medical implant can be constructed of a bioerodible polymer of the types shown and described herein.

In some embodiments, the adhesives described above can be, for example, cement, mucilage, glue, paste, or any other material capable of forming an adhesive bond. Specifically, for example, the adhesives can be constructed of biocompatible polymers or the like. In some embodiments, the electrical conductor is constructed of or includes silicone rubber and the tissue anchor is constructed of or includes polypropylene.

While various embodiments of the invention have been described above, it should be understood that they have been presented by way of example only, and not limitation. Thus, the breadth and scope of the invention should not be limited by any of the above-described embodiments, but should be defined only in accordance with the claims and their equivalents. While the invention has been particularly shown and described with reference to specific embodiments thereof, it will be understood that various changes in form and details may be made. The previous description of the embodiments is provided to enable any person skilled in the art to make or use the invention. For example, a medical device can include various combinations and sub-combinations of the various embodiments described herein.

What is claimed is:

1. An apparatus, comprising:
a coupling portion configured to be coupled to an electrical conductor;
a bioerodible anchor portion adjacent to the coupling portion being moveable from a collapsed configuration to an expanded configuration, the bioerodible anchor portion being configured to anchor the electrical conductor with respect to a body tissue when the bioerodible anchor portion is in its expanded configuration, the bioerodible anchor portion being formulated to erode when disposed within the body tissue at a first rate; and
a bioerodible retainer coupled to the bioerodible anchor portion and configured to inhibit movement of the bioerodible anchor portion from the collapsed configuration to the expanded configuration, the bioerodible retainer being formulated to erode when disposed within the body tissue at a second rate greater than the first rate.

2. The apparatus of claim 1, wherein an axis defined by the bioerodible anchor portion is substantially parallel to an axis defined by the coupling portion when the bioerodible anchor portion is in the collapsed configuration, the bioerodible anchor portion being biased in the expanded configuration.

3. The apparatus of claim 1, wherein the bioerodible anchor portion includes a first tine and a second tine, the first tine being bioerodible at a first rate, the second tine being bioerodible at a second rate different than the first rate.

4. The apparatus of claim 1, wherein the bioerodible anchor portion is configured to release a biocompatible agent as the bioerodible anchor portion bioerodes.

5. The apparatus of claim 1, wherein the coupling portion includes a sleeve configured to be coupled to the electrical conductor via at least one of an interference fit, an adhesive or a weld.

6. The apparatus of claim 1, wherein the coupling portion defines a recess configured to receive an adhesive therein, the adhesive configured to maintain the electrical conductor in a position relative to the coupling portion.

7. An apparatus, comprising:
a retention portion configured to anchor an electrical conductor with respect to a body tissue; and
a coupling portion configured to be coupled to the electrical conductor, the coupling portion defining a lumen therethrough, the lumen configured to receive at least a portion of the electrical conductor, the coupling portion further defining a recess configured to receive an adhesive therein, the adhesive configured to limit movement of the coupling portion relative to the electrical conductor.

8. The apparatus of claim 7, wherein an outer surface of the electrical conductor defines a portion of a boundary of the recess when the electrical conductor is received within the lumen of the coupling portion.

9. The apparatus of claim 7, wherein the coupling portion defines an aperture in fluid communication with the recess.

10. The apparatus of claim 7, wherein the adhesive is a first adhesive, the recess is a first recess, the coupling portion defining a second recess configured to receive a second adhesive therein, the second recess being separate from the first recess.

11. The apparatus of claim 7, wherein the coupling portion defines a first aperture and a second aperture separate from the first aperture, the recess being in fluid communication with the first aperture and the second aperture such that the recess is configured to receive the adhesive when the adhesive is inserted into at least one of the first aperture and the second aperture.

12. The apparatus of claim 7, wherein the recess is configured to receive the adhesive therein such that the adhesive is disposed between the electrical conductor and an inner wall of the coupling portion defining the lumen.

13. The apparatus of claim 7, wherein the recess extends along an axis defined by an inner wall of the coupling portion defining the lumen.

14. The apparatus of claim 7, wherein the recess extends along a circumference of an inner wall of the coupling portion defining the lumen.

15. The apparatus of claim 7, wherein the retention portion is bioerodible, the retention portion being moveable from a collapsed configuration to an expanded configuration, an axis defined by the retention portion being substantially parallel to an axis defined by the coupling portion when the retention portion is in the collapsed configuration, the apparatus further comprising:
a bioerodible coupler configured to maintain the retention portion in its collapsed configuration until the bioerodible coupler substantially erodes after being disposed within the body.

16. An apparatus, comprising:
an electrical conductor including a conductor portion and an eyelet portion; and
a tissue anchor coupled to the electrical conductor, the tissue anchor spaced apart from an outer surface of the electrical conductor, the tissue anchor including a body portion having a first diameter and a retention portion having a second diameter greater than the first diameter, the retention portion being configured to anchor the electrical conductor with respect to body tissue.

17. The apparatus of claim 16, wherein the tissue anchor is one of directly coupled to the eyelet portion or indirectly coupled to the eyelet portion.

18. The apparatus of claim 16, further comprising:
a suture coupled to the eyelet portion, the tissue anchor being coupled to the suture.

19. The apparatus of claim 16, wherein the tissue anchor has a first end portion and a second end portion opposite the first end portion, the first end portion defines a first opening and a second opening, the second end portion defines a third opening and a fourth opening, the tissue anchor defines a first lumen extending from the first opening to the third opening and a second lumen extending from the second opening to the fourth opening,
the eyelet portion being disposed within the first lumen and the second lumen when the tissue anchor is coupled to the eyelet portion.

20. The apparatus of claim 16, wherein the tissue anchor defines a first opening and a second opening, the tissue anchor defines a lumen extending from the first opening to the second opening,
a portion of the eyelet portion being disposed within the lumen when the tissue anchor is coupled to the eyelet portion,
the first opening and an axis defined by the tissue anchor being separated by a first distance, the second opening and the axis defined by the tissue anchor being separated by a second distance, the second distance being less than the first distance.

21. The apparatus of claim 16, further comprising:
a suture coupled to the eyelet portion, the tissue anchor including a coupling portion configured to be coupled to the suture, the coupling portion being substantially spherical in shape.

22. The apparatus of claim 16, the retention portion being bioerodible, the retention portion being moveable from a collapsed configuration to an expanded configuration, an axis defined by the retention portion being substantially parallel to an axis defined by the tissue anchor when the retention portion is in the collapsed configuration, the tissue anchor further comprising:
a bioerodible coupler configured to maintain the retention portion in its collapsed configuration until the bioerodible coupler substantially bioerodes.

23. The apparatus of claim 16, wherein the tissue anchor defines a recess configured to receive an adhesive therein, the adhesive configured to maintain the electrical conductor in a position relative to the tissue anchor.
